(19) United States
(12) Patent Application Publication
(10) Pub. No.: US 2009/0198291 A1
(43) Pub. Date: Aug. 6, 2009

(54) BONE SCREW

(75) Inventors: Foley Kevin, Germantown, TN (US); Gary S. Lindemann, Collierville, TN (US); Newton H. Metcalf, Memphis, TN (US); Greg C. Marik, Collierville, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/423,951

(22) Filed: Apr. 15, 2009

Related U.S. Application Data
(63) Continuation-in-part of application No. 11/586,915, filed on Oct. 26, 2006.

(51) Int. Cl. A61B 17/86

(52) U.S. Cl. 606/305; 606/301

(57) ABSTRACT

A bone screw and method of inserting a bone screw into a bone is disclosed. In one example, the bone screw includes a tip segment for creating a starter hole in the bone. A pilot segment is located proximally of the tip segment for creating a pilot hole in the bone after creation of the starter hole. The starter hole and pilot hole are created by a longitudinal pushing force exerted on the bone screw by the surgeon. A threaded segment is located proximally of the pilot segment for fixedly securing the bone screw in the bone by the use of a rotational force exerted on the bone screw by the surgeon. A head is located proximally of the threaded segment for allowing the bone screw to affix an implant or other soft tissue to the bone. In one embodiment the implant is a plate.

This application is a continuation-in-part of U.S. patent application Ser. No. 11/586,915 filed Oct. 26, 2006.

### TECHNICAL FIELD

The present invention relates generally to bone screws, and more particularly to a bone screw that does not require preparation of a bone or bone segment prior to insertion of the bone screw into the bone.

### BACKGROUND

Bone screws have been used in spinal instrumentation since at least as early as the 1960s. A bone screw is a screw that is usually made of titanium or other metals. In some cases it may be possible to make bone screws out of various synthetic materials as well. If a bone screw is used in a pedicle of the spine, then it is called a pedicle screw. Pedicle screws are most often used as part of a system of screws, plates and rods that immobilize part of the spine. Often times bone screws are used during anterior fixation or plating of cervical vertebral bodies. In this case, the spine is approached from an anterior or antero-lateral direction and bone screws are typically used to solidly mount a spinal plate to the affected vertebrae. The plate used may be a rigid or dynamic plate and can be made from titanium or other metals or various flexible polymers. These types of spinal instrumentation help spinal fusion wherein two or more vertebrae are encouraged to grow together after damage to the intervening disks or spinal fracture makes the original structure unstable.

Current bone screws require multiple steps in order to implant the screw in the spinal bones. Typically, these steps include creating a starting hole with a very small drill bit, pedicle or bone probe, or awl. Once the starter hole is created, a bigger drill bit or pedicle probe may be used to create a pilot hole. After the pilot hole has been created, the pilot hole may then be tapped in a size compatible with the threads of the bone screw to be inserted into the bone. Each additional step required during a surgical procedure to create a hole for a bone screw creates a greater potential for the patient to experience complications from the procedure as well as the chances for a physician to make a mistake due to fatigue during the installation of long spinal constructs requiring numerous bone screws.

Some bone screws are self-tapping thereby eliminating the step of tapping the pilot hole. Some bone screws include a drill-bit feature associated with a thread and shaft; however, using the tip of the screw to drill the hole in the bone can cause a “walking” effect when turning the screw at a speed necessary to drill a hole in the bone much as occurs with a regular drill bit when trying to drill a hole in any hard surface. The tip of the drill bit will move along or “walk” along the surface prior to beginning to drill a hole in the surface. This then creates a situation where the screw can be installed in the wrong position requiring the surgeon to remove the screw and begin the process of installing the screw again.

A similar problem exists when using bone screws in minimal access surgical techniques. Due to the fact of working through a small incision it is difficult to visualize the bone while trying to drill a hole, tap the hole, and then insert a bone screw into a bone segment. In these procedures, a guide wire would typically be inserted first to the location adjacent the bone segment where a bone screw was desired to be installed by the surgeon. The bone drill, tap and bone screw would all be cannulated to allow each of them to be advanced over the guide wire. However, it is difficult to hold the guidewire in place while completing all of the bone preparation procedures and, therefore, it would be desirable to have a bone screw that could be used to complete the creation of the pilot hole, thread formation and threaded installation of the bone screw into the bone segment in one easy step without the use of a guide wire. Furthermore, there are some inherent difficulties when using a guidewire in surgery in close proximity to the spinal column or other delicate anatomies.

Thus, there remains a need for improved, inexpensive, and easy to use bone screws and methods for inserting bone screws in bone that minimize the need for preparatory steps prior to insertion of the bone screws into the bone.

### SUMMARY

A bone screw is disclosed that is capable of directly being inserted into a bone without the requirement of separately creating a starter hole, or a pilot hole and/or tapping the pilot hole. In one embodiment, the bone screw includes a distal tip segment, an intermediate pilot segment, an intermediate threaded segment, and a proximal head or fixation segment. The distal tip segment is used to create a starter hole in the bone. The intermediate pilot segment is used to create a pilot hole in the bone. Typically, the starter hole and pilot hole are created by a longitudinal or axial pushing or hammering of the distal tip on the bone screw into the bone. The intermediate threaded segment of the bone screw is used to fixedly secure the bone screw into the bone. In alternative embodiments, the distal tip segment and the intermediate pilot segment may be combined into a pilot segment which, in essence, eliminates the starter hole segment. This embodiment may be useful for procedures in which a starter hole is not necessary. Again, it is important to note that the creation of the pilot hole is accomplished by longitudinal or axial, not rotational, force on the bone screw. Once the pilot hole is created in the bone segment, the bone screw is then threaded into the bone using a rotational force on the bone screw.

The fixation segment is used as an anchor for a plate, rod or the like that is utilized as part of an immobilization system for a spine. In one embodiment, the fixation segment comprises a head that has a flat top and conical shoulder for securing a plate to the spinal bone segments. In another embodiment, the fixation segment may comprise a head having a rounded top with a flat shoulder under the head. In both embodiments, the head will have either slotted, Philips, Allen, square or other indentation for receiving an installation tool such as a screwdriver or the like. It is envisioned that the bone screw disclosed herein may be used for other types of surgical procedures beyond spinal procedures such as, implant or replacement procedures for example, and as such, may include other head configurations. In another embodiment, the bone screw may be a pedicle screw wherein the fixation segment is a multi-axial or fixed head configuration such that it could receive a rod for fixation to the head of the bone screw.

The tip segment of the bone screw comprises a conical shaped portion that comprises an awl having a sharp point. In another embodiment, the tip segment may include a cutout that forms a cutting edge in the tip segment. As set forth above, the tip segment is used to create a starter or small hole in the bone prior to creation of a pilot hole by using a longitudinal or axial pushing force on the bone screw. The pilot segment comprises an elongate shaft that may be tapered such that it increases in size as the pilot segment enters the bone, but in other embodiments it may not be tapered.

The threaded segment is used to fixedly secure the bone screw in place in the bone. The threaded segment includes a thread that is used to secure the bone screw in the bone by using a rotational turning force on the bone screw. In one embodiment, the thread is formed as a self-tapping thread so that the pilot hole does not need to be tapped. In another embodiment, the threaded segment includes a self-tapping feature as well as threads. The self-tapping features taps the pilot hole prior to the threads of the threaded segment being inserted into the threads created in the bone by the self-tapping feature. In another embodiment, it may be desirable to provide a relatively coarse or cortical bone thread form. And, in another embodiment, it may be desirable to provide a bone screw with a self-tapping feature and then a cancellous bone thread form and then a cortical bone thread form particularly when using a pedicle bone screw. As such, the bone screw disclosed herein is capable of providing three functions that include creating a starter hole, creating a pilot hole, and creating a threaded connection that fixedly secures the bone screw in the bone. This eliminates the need for a surgeon to perform three separate steps in order to insert a bone screw into a bone.

When the bone screw is being used to hold a spinal plate in place, the plate being used may also be made of a polymer that is somewhat flexible and may not even have pre-drilled holes provided in the plate. In this situation, it would be desirable that the bone screw is able to provide the starter hole and pilot hole through the plate without the use of other instrumentation as discussed above.

Another aspect includes a method of inserting a bone screw through a plate having pre-drilled holes and into a bone. In this embodiment and all of the further alternate embodiments discussed below, a starter hole is formed in the bone with a tip segment of the bone screw by use of a longitudinal pushing force on the end of the bone screw. Once the starter hole is formed, a pilot hole is formed in the bone with a pilot segment of the bone screw by continued longitudinal or axial pushing force on the end of the bone screw. The longitudinal or axial pushing force is typically accomplished by the use of hand force by the surgeon, however, a mallet or hammer could also be used to exert pressure on the end of the bone screw. Finally, a threaded connection in the bone is formed with a threaded segment of the bone screw proximate the pilot segment by use of a rotational force on the bone screw through use of a screw driver or the like such that the bone screw is fixedly secured through the plate and into the bone.

Another aspect includes a method of inserting a bone screw through a non-metallic plate having no pre-drilled holes and into a bone. In this embodiment, a starter hole is formed in the plate and then into the bone with a tip segment of the bone screw. Once the starter hole is formed, a pilot hole is formed in the plate and then the bone with a pilot segment of the bone screw. Finally, a threaded connection through the plate and into the bone is formed with a threaded segment of the bone screw proximate the pilot segment such that the bone screw fixedly secures the plate and the bone screw into the bone.

Another aspect includes a method of inserting a bone screw through a torn ligament or muscle or other soft tissue to reattach such tissue to the bone or to other tissue. In this embodiment, the bone screw would be placed into position adjacent such ligament, muscle or other soft tissue and bone. The bone screw would be pushed through such tissue and bone to make a starter hole with the tip of the bone screw. Once the starter hole is formed, a pilot hole is formed in the tissue and bone by continued longitudinal or axial pushing of the bone screw by the surgeon there through. Finally, when the threads of the bone screw come into contact with the tissue and bone, a rotational force is exerted on the bone screw by the surgeon will create a threaded connection between such tissue and bone to securely affix the tissue and bone together. The longitudinal or axial pushing force can be exerted either solely by the surgeon's hand or a hammer or mallet can be used to exert additional force upon the bone screw.

Another aspect includes a method of inserting a plate into the spine across two vertebrae using bone screws according to the present invention. In this embodiment, a plate is placed into position adjacent the bone and a starter hole is formed in the bone through the plate with a tip segment of each of the bone screws. Once the starter hole is formed, a pilot hole is formed in the bone with a pilot segment of each of the bone screws. Finally, a threaded connection in the bone is formed with a threaded segment of each of the bone screws proximate the pilot segment such that the bone screws are fixedly secured in the bone to securely fasten the plate to the vertebrae.

Another aspect includes a method of inserting a flexible implant into the spine across two vertebrae using bone screws according to the present invention. In this embodiment, a flexible implant is placed into position adjacent the bone and a starter hole is formed through the implant and into the bone through the implant with a tip segment of each of the bone screws. Once the starter hole is formed, a pilot hole is formed in the bone with a pilot segment of each of the bone screws. Finally, a threaded connection in the bone is formed with a threaded segment of each of the bone screws proximate the pilot segment such that the bone screws are fixedly secured through the implant and into the bone to securely fasten the implant to the vertebrae.

Another aspect includes a method of inserting an implant into the spine across two vertebrae in a minimal and/or percutaneous access surgery using bone screws according to the present invention. In this embodiment, an implant is placed into position adjacent the bone via a minimal access surgery device and/or percutaneously and each of the fastening screws are introduced into the implant through a minimal access surgery device and/or percutaneously and starter holes are formed in the bone through the implant with a tip segment of each of the bone screws. Once the starter hole is formed, a pilot hole is formed in the bone with a pilot segment of each of the bone screws. Finally, a threaded connection in the bone is formed with a threaded segment of each of the bone screws proximate the pilot segment such that the bone screws are fixedly secured in the bone to securely fasten the implant to the vertebras.

Another aspect includes a method of inserting a rod implant into the spine across two vertebrae using bone screws according to the present invention. In this embodiment, a pedicle screw of the present invention is placed in each vertebra, the pedicle screw having a head configured to receive a rod therein. Once the pedicle bone screws are securely fastened to each vertebra, the rod is affixed within the head of each pedicle bone screw to securely affix the two vertebrae together.

Other systems, methods, features and advantages of the invention will be, or will become apparent to one with skill in the art upon examination of the following figures and detailed description. It is intended that all such additional systems, methods, features and advantages be included within this description, be within the scope of the invention, and be protected by the following claims.

### BRIEF DESCRIPTION OF THE DRAWINGS

The components in the figures are not necessarily to scale, emphasis instead being placed upon illustrating the principles of the invention. Moreover, in the figures, like reference numerals designate corresponding parts throughout the different views.

FIG. 1 is a perspective view of a representative bone screw.

FIG. 2 illustrates the bone screw of FIG. 1 being used with a plate and making a starter hole in a bone of a spine.

FIG. 3 illustrates the bone screw of FIG. 1 making a pilot hole in the bone.

FIG. 4 illustrates the bone screw of FIG. 1 being threaded in to the bone.

FIG. 5 illustrates a bone screw having a rounded head with a flat shoulder under the rounded head.

FIG. 6 illustrates a bone or pedicle screw having a fixation segment with a multi-axial tulip style head configured to receive a spinal rod therein.

### DETAILED DESCRIPTION

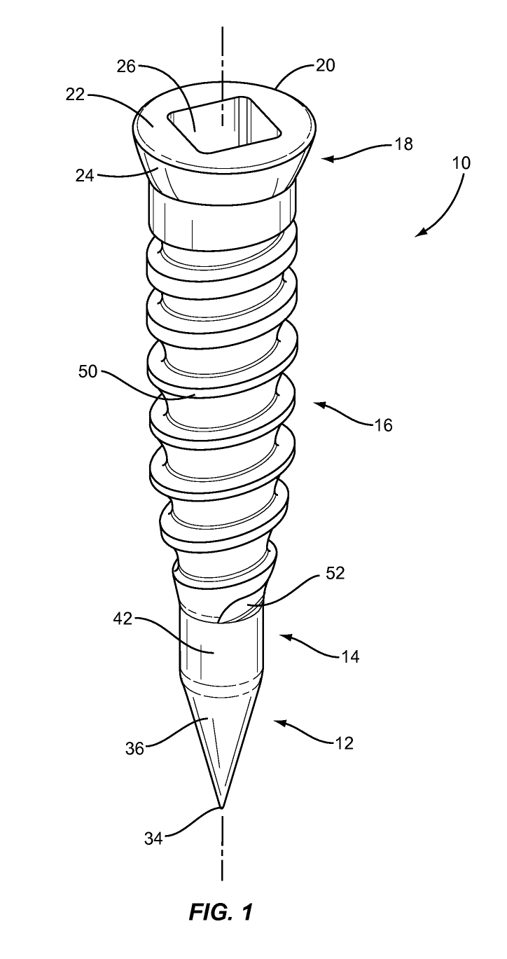

Referring to FIG. 1, a representative bone screw 10 is illustrated that extends along a longitudinal axis and includes a distal tip segment 12, an intermediated pilot segment 14, an intermediate threaded segment 16, and a proximal fixation segment 18. As used herein, distal refers to the direction toward which the bone screw 10 is advanced as the bone screw 10 is engaged to the bone and proximal refers to the direction opposite the distal direction. The fixation segment 18 is shown having a head 20 that has a flat top 22 and conical shoulder 24 for securing a plate or other spinal implant to a bone or bone segment. An indention 26 is provided in the top of the bone screw head 20 for the insertion of a driver (not shown) to install the bone screw 10 into the bone. This indention 26 can take the shape of a square, Allen, Philip, slotted, hexalobular or have any other appropriate geometry to allow for a driver to be used to install the bone screw 10 into the bone.

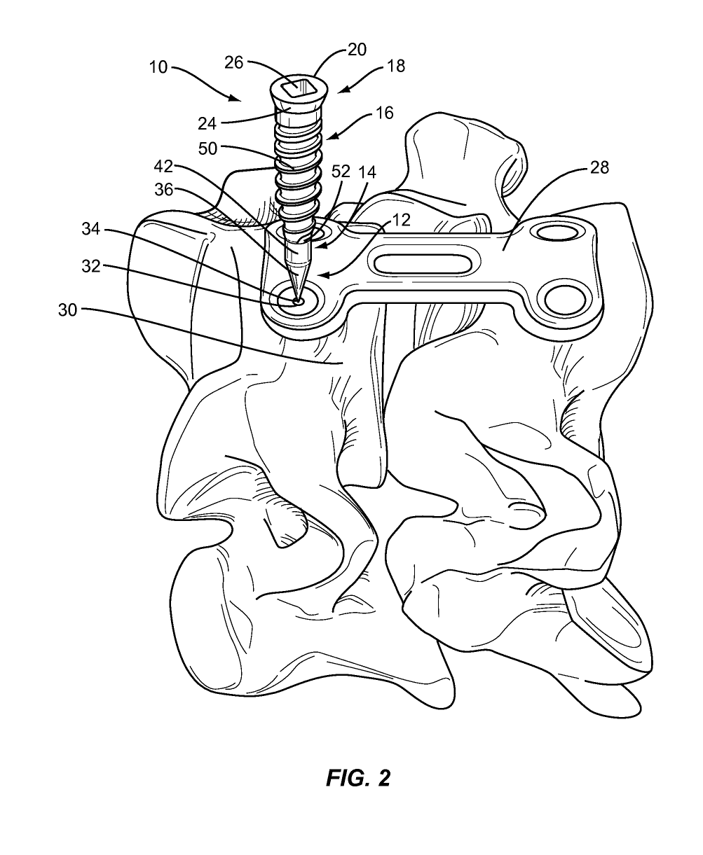

Referring collectively to FIGS. 1-4 and specifically starting with FIG. 2, the bone screw 10 is shown in position above a plate 28 to be affixed to a bone 30 of the spine. The tip segment 12 is used to create a small starter hole 32 or to enlarge an existing starter hole 32 in a bone 30 by the use of a longitudinal or axial pushing force on the proximal end of the bone screw, which in one example comprises a vertebra in which the bone screw 10 is to be inserted through a plate 28 and into the bone 30. In this embodiment, the tip segment 12 includes a tip 34 and a conical portion 36 that gradually increases in size or diameter as it runs up the conical portion 36 toward the pilot segment 14. As such, using the bone screw 10 disclosed herein eliminates the need to create a starter hole with a separate instrument and as a separate step during a surgical procedure.

As set forth in greater detail below, the bone screw 10 is operable to create a starter hole, a pilot hole, and to fixedly secure the bone screw 10 in a bone of a patient with threads. This saves the surgeon time and shortens the length of surgical procedures involving placement of multiple bone screws 10 such as through a plate 28 being affixed to the vertebra of the spine. As a result, the patient does not have to spend as much time undergoing a surgical procedure and the stress experienced by physicians during long procedures is reduced. In other embodiments, creation of a starter hole may not be necessary and as such, only a pilot segment 14 may be included in these embodiments of the bone screw 10. In these embodiments, the tip segment 12 and the pilot segment 14 may be viewed as one and the same.

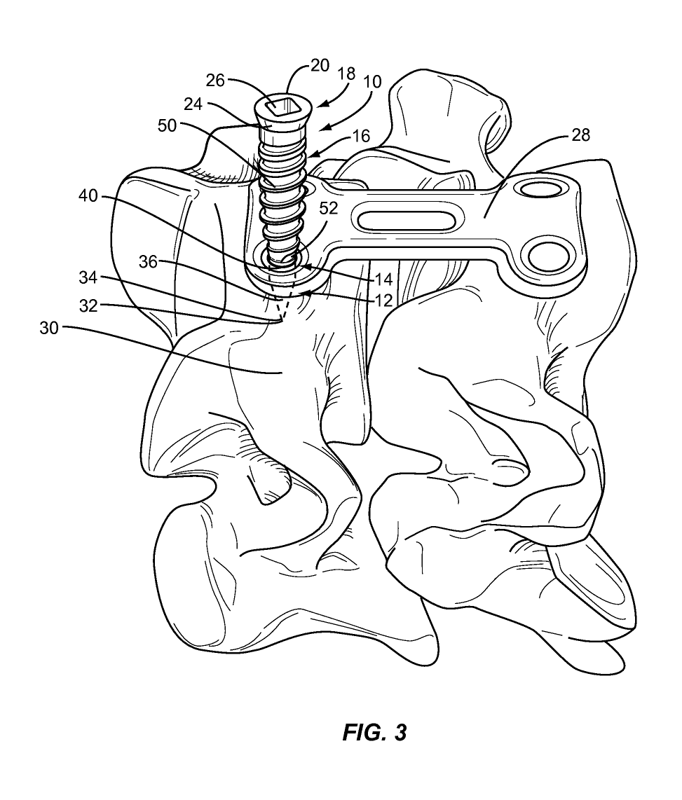

The tip segment 12 transitions into a pilot segment 14 that is located proximate to the tip segment 12. The pilot segment 14 is used to create a pilot hole 40 in the bone 30 after the starter hole 32 has been formed by the tip segment 12, as shown in FIG. 3. The pilot segment 14 includes an elongate cylindrical portion 42. The cylindrical portion 42 may slightly increase in diameter or be tapered (not shown) as it approaches the threaded segment 16. As such, the pilot segment 14 is operable to form a pilot hole 40 in the bone 30 by the use of a longitudinal or axial pushing force on the proximal end of the bone screw. This eliminates the need for a surgeon to utilize a second instrument to create a pilot hole 40 after the starter hole 32 has been created in the bone 30, which eliminates a surgical step.

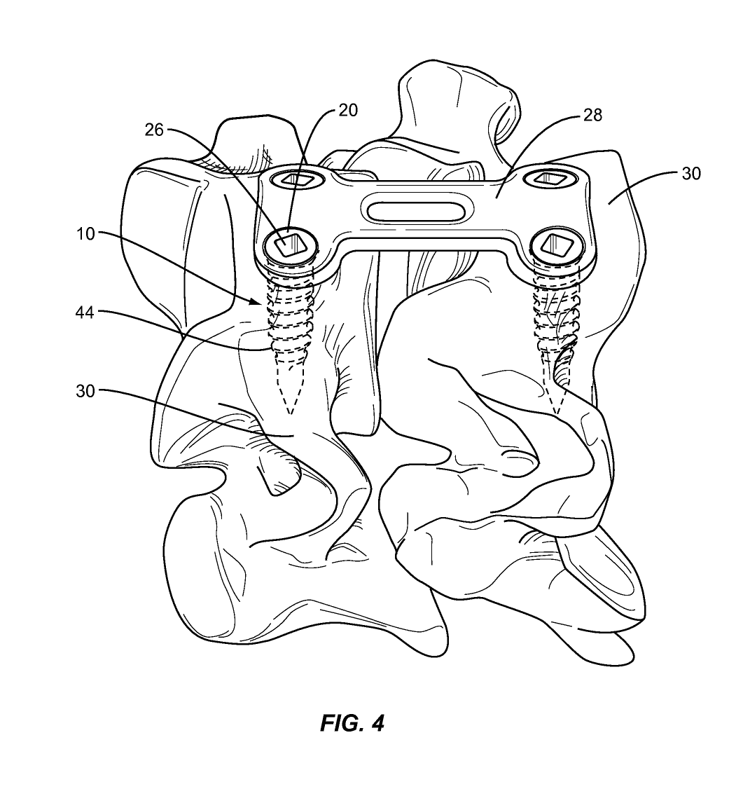

Referring now to FIG. 4, the pilot segment 14 transitions into a threaded segment 16 that is located proximate to the pilot segment 14. The threaded segment 16 is used to fixedly secure the bone screw 10 in the bone 30. The threaded segment 16 has a helically wound, radially outwardly extending bone implantable thread 50 axially extending from the end of the pilot segment 14 to approximately the fixation segment 18. The threaded segment 16 may also include a self-tapping feature 52 located at the distal end of the threaded segment 16. The self-tapping feature 52 cuts a thread in the inside surface of the pilot hole 40 so that the threads 50 of the threaded segment 16 engage or mate with the threads cut in the pilot hole 40. Typically, the threads 50 would be a cortical thread form to securely fasten the bone screw 10 into the cortical portion of the bone 30.

As set forth above, the threaded segment 16 transitions into a fixation segment 18 that is located proximate to the threaded segment 16. In one embodiment, shown in FIGS. 1-4, the fixation segment 18 comprises a head 20 that has a flat top 22 and conical shoulder 24 for securing a plate or other spinal implant to a bone or bone segment. An indention 26 is provided in the top of the bone screw head 20 for the insertion of a driver (not shown) to install the bone screw 10 into the bone. This indention 26 can take the shape of a square, Allen, Philip, slotted, hexalobular or any other appropriate geometry to allow for a driver to be used to install the bone screw 10 into the bone.

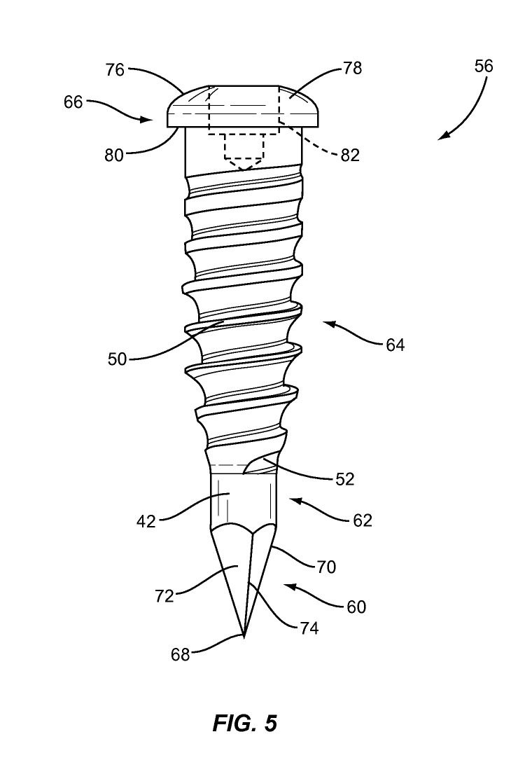

Referring to FIG. 5, in another embodiment, the bone screw 56 shows a tip segment 60, a pilot segment 62, a threaded segment 64, and a fixation segment 66. The tip segment 60 includes a tip 68 and a conical portion 70 that includes a cutout 72 defining a cutting edge 74 that gradually increases in size or diameter as it runs up the conical portion 70 towards the pilot segment 62. As discussed above, to create the starter and pilot holes a longitudinal pushing force would be used to push the tip segment 60 and pilot segment 62 into a bone. In this embodiment, the fixation segment 66 includes a head 76 having a rounded top 78 and having a flat shoulder 80 provided on the underside of the head 76. An indentation 82 may be formed in rounded head 78 of the bone screw 58 for applying torque to bone screw 56 to position it through the plate 28 and into the bone 30 of FIGS. 2-4.

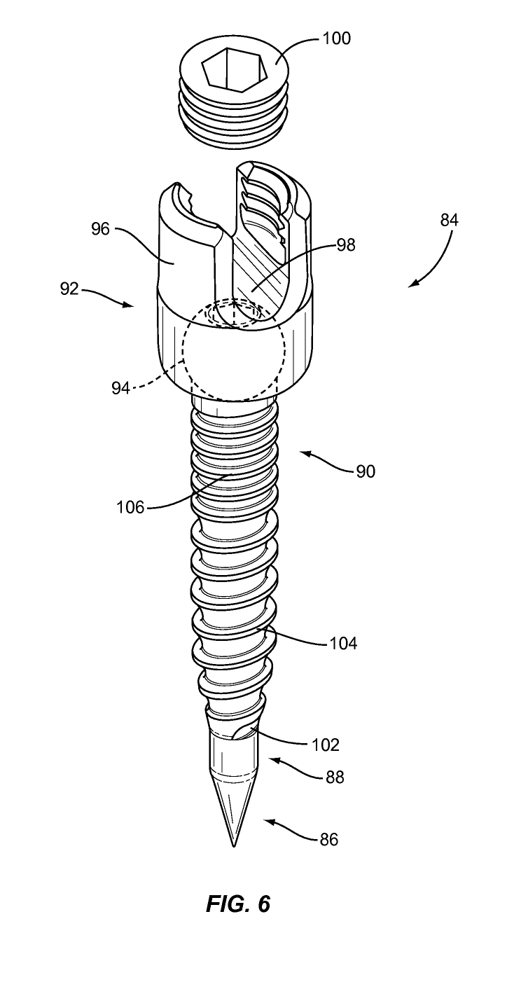

Referring to FIG. 6, in yet another embodiment, a bone screw 84 is shown. Bone screw 84 has a tip segment 86, a pilot segment 88, and an intermediate threaded segment 90. The intermediate threaded segment 90 would include a distally positioned self tapping thread form 102 immediately adjacent the pilot segment 88. Proximately adjacent the self tapping thread form 102 is a cancellous thread form 104, and adjacent the cancellous thread form 104 is a cortical thread form 106. A fixation segment 92 is provided wherein the proximal end of the bone screw 84 is provided with a ball 94. The bone screw 84 is additionally provided with a cylindrically shaped head 96 positioned about the ball 94 such that the head 96 can rotate about the ball 94. The head 96 is provided with a rod receiving slot 98. This type of bone screw 84 is known as a pedicle screw in the industry and would be used with several other pedicle screws to secure a rod (not shown) to the vertebrae. The rod would be secured within the rod receiving slot 98 via a set screw 100 as is well known in the art. It is desirable to use a pedicle screw having the tip segment, pilot segment and intermediate threaded segment of the present invention because it is often times difficult to appropriately visualize the bone site during surgery.

Referring to FIGS. 1-6, another aspect of the present invention relates to a method of inserting a bone screw 10, 56, 84 into a bone 30. The method comprises the steps of (a) forming a starter hole 32 with a tip segment 12, 60, 86 of the bone screw 10, 56, 84 by using longitudinal or axial pushing force on the bone screw by the surgeon; (b) forming a pilot hole 40 with a pilot segment 14, 62, 88 of the bone screw 10, 56, 84 proximate the tip segment 12, 60, 86 by using longitudinal or axial pushing force on the bone screw by the surgeon; and (c) forming a threaded connection 44 in the bone 30 with a threaded segment 16, 64, 90 of the bone screw 10, 56, 84 proximate the pilot segment 14, 62, 88 by the use of a rotational force on the bone screw by the surgeon such that the bone screw 10, 56, 84 is fixedly secured in the bone 30. The longitudinal pushing force exerted by the surgeon can be by means of simple pushing on the proximal end of the bone screw with a screw holder (not shown). However, in some cases this may not exert sufficient force to allow the bone screw to create the starter and pilot holes in the bone. The surgeon may use a hammer to tap the bone screw in place in the bone. When the threaded segment 16, 64, 90 of the bone screw 10, 56, 84 reaches the bone, the surgeon would then exert a rotational force on the bone screw by use of an appropriate driver (not shown) to properly install the bone screw to the bone.

In this fashion, the bone screw of the present invention can also be used to reattach a ligament, muscle or other soft tissue to the bone. The method comprises the steps of (a) positioning the ligament, muscle or other soft tissue adjacent the bone intended for reattached to; (b) forming a starter hole in the tissue and bone with a tip segment 60 of the bone screw 56 by using a longitudinal or axial pushing force on the bone screw by the surgeon; (c) forming a pilot hole in the tissue and bone with a pilot segment 62 of the bone screw 56 by continued longitudinal or axial pushing force on the bone screw by the surgeon; and (d) forming a threaded connection in the bone with a threaded segment 64 of the bone screw 56 by use of a rotational force on the bone screw by the surgeon such that the bone screw securely attaches the soft tissue to the bone. The longitudinal or axial pushing force by the surgeon can be aided by use of a hammer or mallet and the rotational force would typically be accomplished by a driver, like a screwdriver, utilized by the surgeon.

While the invention has been illustrated and described in detail in the drawings and foregoing description, the same is to be considered as illustrative and not restrictive in character.

What is claimed is:

1. A bone screw comprising:
a distal tip segment for creating a starter hole in a bone;
a pilot segment proximate said distal tip segment for creating a pilot hole in said bone after creation of said starter hole;
a threaded segment proximate said pilot segment for fixedly securing said bone screw in said bone; and
a fixation segment proximate said threaded segment.

2. The bone screw of claim 1, wherein said fixation segment may comprise a head having a top and a conical shoulder adjacent said top, the head further having an indentation in its top surface for receiving an installation tool for affixing the bone screw to the bone.

3. The bone screw of claim 1, wherein said fixation segment may comprise a ball on the proximal end of the bone screw and having a cylindrical head positioned about said ball, the cylindrical head having a rod receiving slot through its proximal end, and further having a set screw threadably attachable to the proximal end of the cylindrical head to capture a rod within the rod receiving slot.

4. The bone screw of claim 1, wherein said tip segment comprises an awl having a sharp point such that upon a longitudinal pushing force on the bone screw a starter hole is formed in the bone.

5. The bone screw of claim 4, wherein said tip segment comprises an awl that transitions into a conical portion, the conical portion having a cutting edge provided thereon such that upon a longitudinal pushing force on the bone screw a pilot hole is formed in the bone.

6. The bone screw of claim 1, wherein said threaded segment includes a self-tapping thread form positioned distally adjacent a cortical thread form such that upon a rotational force being exerted on the bone screw the self-tapping thread form creates threads in the bone and the cortical thread form solidly affixes the bone screw into the bone.

7. The bone screw of claim 1, wherein the bone screw secures an implant to the bone.

8. A method of inserting a bone screw through an implant and into a bone, comprising:
forming a starter hole with a distal tip segment of said bone screw by use of a longitudinal pushing force exerted on the bone screw; and
forming a pilot hole with a pilot segment of said bone screw proximate said tip segment by use of a longitudinal pushing force on the bone screw; and
forming a threaded connection in said bone with a threaded segment of said bone screw proximate said pilot segment by use of a rotational force on the bone screw such that said bone screw is fixedly secured in said bone thereby securing the implant to the bone.

9. The method of claim 8, wherein said tip segment comprises an awl.

10. The method of claim 8, wherein said tip segment comprises a conical shaped portion having a cutout forming a cutting edge.

11. The method of claim 9, wherein said threaded segment has a cortical thread form.

12. The method of claim 9, where said threaded segment has a distally positioned self-tapping thread form and an adjacent cortical thread form.

13. The method of claim 8, wherein said implant is a plate.

14. A method of inserting a bone screw into a bone, comprising:
(a) forming a starter hole in said bone with a tip segment of said bone screw by use of a longitudinal pushing force exerted on the bone screw;
(b) forming a pilot hole with a pilot segment of said bone screw by use of a longitudinal pushing force on the bone screw;
(c) forming a threaded connection in said bone with a threaded segment of said bone screw proximate said pilot segment by use of a rotational force on the bone screw such that said bone screw is fixedly secured in said bone thereby securing the implant to the bone; and
wherein steps (a), (b), and (c) above are performed by said bone screw in one fluid surgical step.

15. The method of claim 14, wherein said threaded segment includes a self-tapping thread form proximate said threaded segment, the threaded segment having a cortical thread form.

16. The method of claim 15, wherein the threaded segment has a cancellous thread form positioned between the self-tapping thread form and the cortical thread form.

17. The method of claim 14, wherein said bone screw is used to affix an implant to the bone, said implant being a plate wherein the plate is first positioned adjacent the bone and the bone screw is inserted through the plate and into the bone to affix the plate to the bone.

18. The method of claim 14, wherein said bone screw is used to affix soft tissue to the bone, the soft tissue being first positioned adjacent the bone and the bone screw being inserted through the soft tissue and into the bone to affix the soft tissue to the bone.

19. The method of claim 14, wherein said bone screw is used to affix an implant to the bone, said implant being a rod and wherein steps (a), (b), and (c) above are first performed by said bone screw and then the bone screw is affixed to said rod.

20. The method of claim 17, wherein said plate is a flexible plate without predrilled holes such that step (a) includes forming a starter hole in said plate; and step (b) includes forming a pilot hole in said plate.
